(19) United States
(12) Patent Application Publication
(10) Pub. No.: US 2003/0003070 A1
(43) Pub. Date: Jan. 2, 2003

(54) Use of nanoscale active anti-dandruff ingredients

(76) Inventors: Anke Eggers, Duesseldorf (DE); Werner Seipel, Hilden (DE); Hermann Hensen, Haan (DE)

Correspondence Address: COGNIS CORPORATION, 2500 RENAISSANCE BLVD., SUITE 200, GULPH MILLS, PA, 19406

(21) Appl. No.: 10/168,237

(22) PCT Filed: Dec. 11, 2000
(86) PCT No.: PCT/EP00/12519

(30) Foreign Application Priority Data
Dec. 18, 1999 (DE) 19961278.1

(51) Int. Cl. A61K 7/06; B29B 9/00

(52) U.S. Cl. 424/70.1; 264/5

(57) ABSTRACT

The invention relates to nanoscale active anti-dandruff ingredients having a particle diameter ranging from 10-500 nm in the production of hair-cosmetic preparations.

This invention relates generally to nanoparticles and more particularly to the use of nanoscale antidandruff agents in hair-care preparations.

### PRIOR ART

Various hair-care preparations containing antidandruff agents are known from the prior art. However, the effect of these antidandruff agents is always associated with their distribution on the scalp and the rate at which the compounds are incorporated and absorbed. So far as the compounds hitherto available are concerned, there is considerable potential for improvement in this regard.

Accordingly, the problem addressed by the present invention was to accelerate the uptake of antidandruff agents during their application by providing new supply forms. In addition, they would have a long-lasting effect after application and good dermatological compatibility and would be distinguished by excellent stability during storage at elevated temperature.

### DESCRIPTION OF THE INVENTION

The present invention relates to the use of nanoscale antidandruff agents in the 10 to 300 nm range for the production of cosmetic hair-care preparations.

It has surprisingly been found that the stability of cosmetic hair-care preparations, such as rinses and shampoos for example, is increased and their effectiveness is significantly accelerated and improved by the addition of antidandruff agents providing the antidandruff agents are present in the form of nanoparticles, i.e. particles with a mean diameter of 10 to 500, preferably 50 to 300 and more particularly 100 to 150 nm. At the same time, the preparations in question show high dermatological compatibility.

Antidandruff Agents

Suitable antidandruff agents are Piroctone Olamine (1-hydroxy-4-methyl-6-(2,4,4-trimethylpentyl)-2-(1H)-pyridinone monoethanolamine salt), Baypival® (climbazole), Ketoconazole® (4-acetyl-1-{4-[2-(2,4-dichlorophenyl) r-2-(1H-imidazol-1-ylmethyl)-1,3-dioxylan-c-4-ylmethoxyphenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid monoethanolamide sulfosuccinate Na salt, Lamepon® UD (protein/undecylenic acid condensate), zinc pyrithione, aluminium pyrithione and magnesium pyrithione/dipyrithione magnesium sulfate. 1-hydroxy-4-methyl-6-(2,4,4-trimethylpentyl)-2-(1H)-pyridinone monoethanolamine salt, magnesium pyrithione/dipyrithione magnesium sulfate, selenium disulfide, zinc pyrithione and/or climbazole are preferably used.

Production of Nanoparticles

One process for the production of nanoparticles by rapid expansion of supercritical solutions (RESS process) is known from the article by S. Chihlar, M. Türk and K. Schaber in Proceedings World Congress on Particle Technology 3, Brighton, 1998. A preferred embodiment of the invention is characterized by the use of nanoscale antidandruff agents obtained by

(a) dissolving the starting materials in a suitable solvent under supercritical or near-critical conditions,

(b) expanding the fluid mixture through a nozzle into a vacuum, a gas or a liquid and

(c) simultaneously evaporating the solvent.

To prevent the nanoparticles from agglomerating, it is advisable to dissolve the starting materials in the presence of suitable protective colloids or emulsifiers and/or to expand the critical solutions into aqueous and/or alcoholic solutions of the protective colloids or emulsifiers or into cosmetic oils which may in turn contain redissolved emulsifiers and/or protective colloids. Suitable protective colloids are, for example, gelatine, casein, gum arabic, lysalbinic acid, starch and polymers, such as polyvinyl alcohols, polyvinyl pyrrolidones, polyalkylene glycols and polyacrylates. Accordingly, the nanoscale antidandruff agents preferably used are those which are surrounded by a protective colloid and/or an emulsifier. The protective colloids or emulsifiers are normally used in quantities of 0.1 to 20% by weight and preferably in quantities of 5 to 15% by weight, based on the antidandruff agents.

Another suitable process for the production of nanoscale particles is the evaporation technique. Here, the starting materials are dissolved in a suitable organic solvent (for example alkanes, vegetable oils, ethers, esters, ketones, acetals and the like). The resulting solutions are then introduced into water or another nonsolvent—optionally in the presence of a surface-active compound dissolved therein—so that the homogenization of the two immiscible solvents results in precipitation of the nanoparticles, the organic solvent preferably evaporating. O/w emulsions or o/w microemulsions may be used instead of an aqueous solution. The emulsifiers and protective colloids mentioned at the beginning may be used as the surface-active compounds. Another method for the production of nanoparticles is the so-called GAS process (gas anti-solvent recrystallization). This process uses a highly compressed gas or supercritical fluid (for example carbon dioxide) as non-solvent for the crystallization of dissolved substances. The compressed gas phase is introduced into the primary solution of the starting materials and absorbed therein so that there is an increase in the liquid volume and a reduction in solubility and fine particles are precipitated. The PCA process (precipitation with a compressed fluid anti-solvent) is equally suitable. In this process, the primary solution of the starting materials is introduced into a supercritical fluid which results in the formation of very fine droplets in which diffusion processes take place so that very fine particles are precipitated. In the PGSS process (particles from gas saturated solutions), the starting materials are melted by the introduction of gas under pressure (for example carbon dioxide or propane). Temperature and pressure reach near- or super-critical conditions. The gas phase dissolves in the solid and lowers the melting temperature, the viscosity and the surface tension. On expansion through a nozzle, very fine particles are formed as a result of cooling effects.

Commercial Applications

Compared with known antidandruff agents, the particular fineness of the particles provides for increased stability and consistency of the hair-care preparations. They may be used for the production of cosmetic hair-care preparations. The quantity in which the antidandruff agents are used is normally of the order of 0.01 to 10, preferably 0.2 to 5 and more particularly 0.5 to 2% by weight, based on the preparations.

The cosmetic preparations may also contain mild surfactants, oil components, emulsifiers, superfatting agents, pearlizing waxes, consistency factors, thickeners, silicone compounds, fats, waxes, biogenic agents, deodorants, film formers, swelling agents, antioxidants, hydrotropes, preservatives, solubilizers, perfume oils, dyes and the like as further auxiliaries and additives.

Typical examples of suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines amphoacetals and/or protein fatty acid condensates, preferably based on wheat proteins.

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

Suitable emulsifiers are, for example, nonionic surfactants from at least one of the following groups:

products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

adducts of 1 to 15 moles of ethylene oxide with castor oil and/or hydrogenated castor oil;

adducts of 15 to 60 moles of ethylene oxide with castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide;

partial esters of polyglycerol (average degree of self-condensation 2 to 8), polyethylene glycol (molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 20 24 051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate. (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof.

Examples of other suitable polyolesters are the mono-, di- and triesters of trimethylolpropane or pentaerythritol with lauric acid, coconut fatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers and antiperspirants.

Basically, suitable germ inhibitors are any substances which act against gram-positive bacteria such as, for example, 4-hydroxybenzoic acidand salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane -1,2-diol, 3-iodo-2-propinyl butyl carbamate, chlor-hexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Cognis GmbH, Düsseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

nonaqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (ACosmetics Directive≅).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes as listed, for example, in the publication AKosmetische Färbemittel≅ of the Farbstoffkommission der Deutschen Forschungsgemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### EXAMPLES

To produce the nanoscale metal soaps (Examples 1 to 6), carbon dioxide was first taken from a reservoir under a constant pressure of 60 bar and was purified in a column with an active carbon and a molecular sieve packing. After liquefaction, the CO₂ was compressed to the required supercritical pressure p by a diaphragm pump at a constant delivery rate of 3.5 l/h. The solvent was then brought to the necessary temperature T1 in a preheater and was introduced into an extraction column (steel, 400 ml) charged with the waxes. The resulting supercritical, i.e. fluid, mixture was sprayed through a laser-drawn nozzle (length 830 μm, diameter 45 μm) at a temperature T2 into a Plexiglas expansion chamber containing a 4% by weight aqueous solution of an emulsifier or protective colloid. The fluid medium evaporated, leaving the dispersed nanoparticles encapsulated in the protective colloid behind. The process conditions and the average particle size range (as determined photometrically by the 3-WEM method) are set out in Table 1 below.

TABLE 1
Nanoparticles

| | | Sol- | p | T1 | T2 | Emulsifier/pro- | PSR |
|---|---|---|---|---|---|---|---|
| Ex. | Polymer | vent | bar | ° C. | ° C. | tective colloid | nm |
| 1 | Zinc pyrithione | CO₂ | 200 | 80 | 175 | Polyvinyl alcohol | 60-120 |
| 2 | Octopirox ® | CO₂ | 180 | 70 | 160 | Polyethylene | 75-120 |
| | | | | | | glycol (M = 400) | |
| 3 | Ketoconazole ® | CO₂ | 200 | 85 | 180 | Polyvinyl alcohol | 75-130 |
| 4 | Selenium disulfide | CO₂ | 200 | 85 | 175 | Polyvinyl alcohol | 60-140 |
| 5 | Magnesium bispyrithione | CO₂ | 200 | 85 | 175 | Polyvinyl alcohol | 55-140 |
| 6 | Baypival ® | CO₂ | 200 | 85 | 175 | Polyvinyl alcohol | 60-140 |

In order to test effectiveness, 20 volunteers were treated with antidandruff preparations 1 to 4 according to the invention and with comparison preparations C1 and C2. Their effects were evaluated on a scale of 1 (very good) to 4 (adequate).

TABLE 2
Nanoscale preparations - quantities = % by weight

| Composition/performance | 1 | 2 | 3 | 4 | C1 | C2 |
|---|---|---|---|---|---|---|
| Sodium Laureth Sulfate | 5.5 | 5.5 | 5.5 | 5.5 | 5.5 | 5.5 |
| Ammonium Laureth Sulfate | 2.4 | 2.4 | 2.4 | 2.4 | 2.4 | 2.4 |
| Cocamide DEA | 1.5 | 1.5 | 1.5 | 1.5 | 1.5 | 1.5 |
| Dimethicone | − | 3 | 3 | 3 | − | 2 |
| Zinc pyrithione | − | − | − | − | 0.3 | 2.5 |
| Laureth-2 | 2 | − | 2 | 2 | 2 | 2 |
| Sodium Chloride | 0.2 | 0.3 | 1.0 | 0.5 | 0.5 | 0.5 |
| Zinc pyrithione (nanorized) | 0.3 | − | − | − | − | − |
| Octopirox ® (nanorized) | − | − | 2 | 0.3 | − | − |
| Ketoconazole ® (nanorized) | − | 2.5 | − | 4 | − | − |
| Water | to 100 | | | | | |
| Effectiveness | 1 | 2 | 1 | 1 | 3 | 4 |
| Stability | ++ | ++ | + | ++ | + | − |
| Dermatological compatibility | ++ | ++ | + | ++ | + | + |

Table 3 below contains a number of formulation examples with cationic nanoparticles.

TABLE 3
Hair-care preparations (water, preservative to 100% by weight) -
quantities in % by weight, based on the final concentration.

| Composition (INCI) | 1 | 2 | 3 | 4 | 5 | 6 |
|---|---|---|---|---|---|---|
| Texapon ® NSO | — | — | — | — | — | — |
| Sodium Laureth Sulfate | | | | | | |
| Texapon ® SB 3 | — | — | — | — | — | — |
| Disodium Laureth Sulfosuccinate | | | | | | |
| Plantacare ® 818 | — | — | — | — | — | — |
| Coca Glucosides | | | | | | |
| Plantacare ® PS 10 | — | — | — | — | — | — |
| Sodium Laureth Sulfate (and) Coca Glucosides | | | | | | |
| Dehyton ® PK 45 | — | — | — | — | — | — |
| Cocamidapropyl Betaine | | | | | | |
| Dehyquart ® A | 2.0 | 2.0 | 2.0 | 2.0 | 4.0 | 4.0 |
| Cetrimonium Chloride | | | | | | |
| Dehyquart L ® 80 | 1.2 | 1.2 | 1.2 | 1.2 | 0.6 | 0.6 |
| Dococaylmethylethoxymonium Methosulfate (and) Propyleneglycol | | | | | | |
| Eumulgin ® B2 | 0.8 | 0.8 | — | 0.8 | — | 1.0 |
| Ceteareth-20 | | | | | | |
| Eumulgin ® VL 75 | — | — | 0.8 | — | 0.8 | — |
| Lauryl Glucoside (and) Polyglyceryl-2 Polyhydroxystearate (and) Glycerin | | | | | | |
| Lanette ® O | 2.5 | 2.5 | 2.5 | 2.5 | 3.0 | 2.5 |
| Cetearyl Alcohol | | | | | | |
| Cutina ® GMS | 0.5 | 0.5 | 0.5 | 0.5 | 0.5 | 1.0 |
| Glyceryl Stearate | | | | | | |
| Cetiol ® HE | 1.0 | — | — | — | — | — |
| PEG-7 Glyceryl Cocoate | | | | | | |
| Cetiol ® PGL | — | 1.0 | — | — | 1.0 | — |
| Hexyldecanol (and) Hexyldecyl laurate | | | | | | |
| Cetiol ® V | — | — | — | 1.0 | — | — |
| Decyl Oleate | | | | | | |
| Eutanol ® G | — | — | 1.0 | — | — | 1.0 |
| Octyldodecanol | | | | | | |
| Nutrilan ® Keratin W | — | — | — | 2.0 | — | — |
| Hydrolyzed Keratin | | | | | | |
| Lamesoft ® LMG | — | — | — | — | — | — |
| Glyceryl Laurate (and) Potassium Cocoyl Hydrolyzed Collagen | | | | | | |
| Euperlan ® PK 3000 AM | — | — | — | — | — | — |
| Glycol Distearate (and) Laureth-4 (and) Cocamidopropyl Betaine | | | | | | |
| Generol ® 122 N | — | — | — | — | 1.0 | 1.0 |
| Soya Sterol | | | | | | |
| Hydagen ® HCMF | 1.0 | 1.0 | 1.0 | 1.0 | 1.0 | 1.0 |
| Chitosan | | | | | | |
| Compound 2 according to the invention | 0.3 | — | — | — | — | — |
| Compound 3 according to the invention | — | 0.5 | 2 | 2.5 | — | — |
| Compound 5 according to the invention | — | — | — | — | 0.5 | 4 |
| Copherol ® 12250 | — | — | 0.1 | 0.1 | — | — |
| Tocopherol Acetate | | | | | | |
| Arlypon ® F | — | — | — | — | — | — |
| Laureth-2 | | | | | | |
| Sodium Chloride | — | — | — | — | — | — |

TABLE 4
Hair shampoo preparation (water, preservative to 100% by weight) -
quantities in % by weight, based on the final concentration

| Composition (INCI) | 7 | 8 | 9 | 10 | 11 |
|---|---|---|---|---|---|
| Texapon ® NSO | 11.0 | — | — | — | — |
| Sodium Laureth Sulfate | | | | | |
| Texpon ® K 14 S | — | — | — | 11.0 | 23.0 |
| Sodium Myreth Sulfate | | | | | |
| Texapon ® SB 3 | 7.0 | — | — | — | — |
| Disodium Laureth Sulfosuccinate | | | | | |
| Plantacare ® 818 | — | — | — | 6.0 | 4.0 |
| Coca Glucosides | | | | | |
| Plantacare ® 2000 | 4.0 | — | — | — | — |
| Decyl Glucoside | | | | | |
| Plantacare ® PS 10 | — | 16.0 | 17.0 | — | — |
| Sodium Laureth Sulfate (and) Coca Glucosides | | | | | |
| Dehyton ® PK 45 | — | — | — | — | 7.0 |
| Cocamidopropyl Betaine | | | | | |
| Eumulgin ® B1 | — | — | — | — | — |
| Ceteareth-12 | | | | | |
| Eumulgin ® B2 | — | — | — | — | — |
| Ceteareth-20 | | | | | |
| Lameform ® TGI | — | — | — | — | — |
| Polyglyceryl-3 Isostearate | | | | | |
| Dehymuls ® PGPH | — | — | — | — | — |
| Polyglyceryl-2 Dipolyhydroxystearate | | | | | |
| Monomuls ® 90-L 12 | — | — | — | 1.0 | 1.0 |
| Glyceryl Laurate | | | | | |
| Cetiol ® HE | — | — | — | — | — |
| PEG-7 Glyceryl Cocoate | | | | | |
| Eutanol ® G | — | — | — | — | — |
| Octyldodecanol | | | | | |
| Nutrilan ® Keratin W | — | — | — | 2.0 | 2.0 |
| Hydrolyzed Keratin | | | | | |
| Nutrilan ® I | 2.0 | — | 2.0 | — | — |
| Hydrolyzed Collagen | | | | | |
| Lamesoft ® LMG | — | — | — | 1.0 | — |
| Glyceryl Laurate (and) Potassium Cocoyl Hydrolyzed Collagen | | | | | |
| Lamesoft ® 156 | — | — | — | — | 5.0 |
| Hydrogenated Tallow Glyceride (and) Potassium Cocoyl Hyrolyzed Collagen | | | | | |
| Gluadin ® WK | 1.0 | 2.0 | 2.0 | 2.0 | — |
| Sodium Cocoyl Hydrolyzed Wheat Protein | | | | | |
| Euperlan ® PK 3000 AM | — | — | 3.0 | 3.0 | — |
| Glycol Distearate (and) Laureth-4 (and) Cocamidopropyl Betaine | | | | | |
| Panthenol | — | — | — | — | — |
| Arlypon ® F | — | — | — | — | — |
| Laureth-2 | | | | | |
| Hydagen ® CMF | 1.0 | 1.0 | 1.0 | 1.0 | 1.0 |
| Chitosan | | | | | |
| Compound 1 according to the invention | 4 | — | — | 0.5 | — |
| Compound 4 according to the invention | 1 | 0.2 | 3 | — | 2 |
| Sodium Chloride | 1.6 | 2.0 | 2.2 | — | 3.0 |
| Glycerin (86% by weight) | — | — | 1.0 | 3.0 | — |

(1-4) hair rinse, (5-6) conditioner

1. The use of nanoscale antidandruff agents with particle diameters of 10 to 500 nm for the production of cosmetic hair-care preparations.

2. The use claimed in claim 1, characterized in that antidandruff agents selected from the group consisting of 1-hydroxy-4-methyl-6-(2,4,4-trimethylpentyl)-2-(1H)-pyridinone monoethanolamine salt, climbazole, 4-acetyl-1-{4-[2-(2,4-dichlorophenyl) r-2-(1H-imidazol-1-ylmethyl)-1,3-dioxylan-c-4-ylmethoxyphenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid monoethanolamide sulfosuccinate Na salt, protein/undecylenic acid condensate, zinc pyrithione, aluminium pyrithione and magnesium pyrithione/dipyrithione magnesium sulfate are used.

3. The use claimed in claims 1 and/or 2, characterized in that 1-hydroxy-4-methyl-6-(2,4,4-trimethylpentyl)-2-(1H)-pyridinone monoethanolamine salt, magnesium pyrithione/dipyrithione magnesium sulfate, selenium disulfide, zinc pyrithione and/or climbazole are preferably used.

4. The use claimed in at least one of claims 1 to 3, characterized in that antidandruff agents obtained by
(a) dissolving the starting materials in a suitable solvent under supercritical or near-critical conditions,
(b) expanding the fluid mixture through a nozzle into a vacuum, a gas or a liquid and
(c) simultaneously evaporating the solvent
are used.

5. The use claimed in at least one of claims 1 to 4, characterized in that nanoparticles coated with a protective colloid are used.

6. The use claimed in claim 5, characterized in that polyvinyl alcohol or polyethylene glycol is used as the protective colloid.

7. The use claimed in at least one of claims 1 to 6, characterized in that the antidandruff agents are used in quantities of 0.01 to 10% by weight, based on the cosmetic hair-care preparations.
